(19) United States
(12) Patent Application Publication
(10) Pub. No.: US 2011/0262304 A1
(43) Pub. Date: Oct. 27, 2011

(54) METHOD OF FLUID CONTROL IN MEDICAL DIAGNOSTIC MEDIA

(75) Inventors: James A. Profitt, Goshen, IN (US); Chris T. Zimmerle, Goshen, IN (US)

(73) Assignee: Siemens Healthcare Diagnostics Inc., Tarrytown, NY (US)

(21) Appl. No.: 12/674,941

(22) PCT Filed: Aug. 27, 2008
(86) PCT No.: PCT/US08/74369

§ 371 Date: Feb. 24, 2010

Related U.S. Application Data
(60) Provisional application No. 60/968,397, filed on Aug. 28, 2007.

(51) Int. Cl. G01N 33/48; B29C 35/08

(52) U.S. Cl. 422/68.1; 264/400

(57) ABSTRACT

Migration of liquid samples on diagnostic test strips is prevented by dividing the test strips into reagent-containing pads spaced about 0.3 to 3 mm apart with a laser.

### FIELD OF THE INVENTION

The present invention relates to the field of diagnostic testing and more specifically to creating a physical barrier in a dry reagent to prevent the spread of fluid sample. The invention confines the spread of fluid which occurs upon application of sample to an indicating medium, such as reagent paper for urinalysis. In addition, the invention is a manufacturing method which has potential to save material, production steps, assembly time and manufacturing costs.

### BACKGROUND OF THE INVENTION

When using a dry reagent to test numerous fluid samples, it is important to be able to prevent the fluid samples placed on a designated area of a carrier containing the dry reagent from spreading to other areas containing the dry reagent. The spread of the sample from one area of the dry reagent to another can result in crosstalk or carryover which may yield erroneous readings or sample contamination. A number of solutions to this problem have been proposed in the past.

A reagent card for making multiple analyses of analytes is described in U.S. Pat. No. 4,526,753. Ribbons of impregnated matrix material were applied in parallel on a substrate and then cut into separated matrices along each ribbon. It was suggested that the cuts could be made by several means including a rotary die, a stamp, a punch etc. The formed matrices were said to be sufficient to permit individual analyses “without encountering runover problems with sample and reagent material.” No dimensions were given that would provide the desired results.

In the many years since the '753 patent issued the technology has advanced, introducing new problems not pertinent to the '753 patent. More particularly, the size of the matrices, also referred to as “pads” in the art, has become smaller and scanning of the reagent response has become much more precise. While the color or other optically recognized reagent response was previously read optically as an average value across the entire reagent-containing pad, it is now possible to measure the response of a reagent at each one of many subdivisions of the pad. For example, a region measuring 0.11 to 0.18 inches (2.8 to 4.6 mm) within a reagent-containing pad measuring 0.13 to 0.2 inches (3.3 to 5.1 mm) can be divided into 875 sub-regions, each of which can be measured by red, green and blue light response by the photosensitive elements of a CCD or CMOS array. Thus, providing accurate measurements has become more complex, while at the same time making possible better results.

Siemens Healthcare Diagnostics Inc CLINITEK ATLAS® Automated Urine Chemistry Analyzer system uses a roll of film upon which plastic strips have been attached, each of them containing multiple 0.2×0.2 inch (5.1×5.1 mm) pads of dry diagnostic reagent. Patient samples are transferred by the instrument's pipette and applied in a predetermined volume of each of the pads, saturating the pad with sample fluid. The strips on the roll of the CLINITEK ATLAS® assay material reduce or prevent crosstalk or carryover by adequately spacing the strips and pads from one another, for example leaving a lateral space of at least 0.5 inches (13 mm) Other solutions to the crosstalk problem include loading individual strips and dipping them into sample fluid. This method prevents carryover by only testing one patient sample at a time, but it requires more sample, more testing time and more handling.

There are a number of disadvantages with the above-mentioned solutions. To assure that sample cross-talk or carryover do not occur, strips on the roll of the CLINITEK ATLAS® assay material must be well separated, making the rolls rather long and limiting the number of samples which can be analyzed before loading a new roll. Systems which handle strips individually are at a disadvantage for design improvement when further miniaturization is attempted, as strips become more challenging to handle when they are smaller. A further disadvantage is that more sample fluid is required, meaning more waste fluid must be handled by the instrument system.

When many measurements are to be made on a small reagent-containing pad the edges of the pad become more important than was previously the case. Many pads are made of fibrous materials, such as filter papers. Ideally the edges should be precisely formed, with no fibers extending beyond the edge. Otherwise, liquid may be induced to migrate between adjacent pads, causing inaccurate measurement of the regions adjacent the edges. Consequently, cutting a strip of reagent-containing fibrous material has become more difficult as advancing technology has miniaturized the system. Typically, the pads are attached to an underlying carrier by double stick adhesives. Among the ways to cut fibrous materials to size are mechanical methods which use the cutting action of one metal part against another to slice the materials. Disadvantages include dust generation, adhesive buildup on the cutting edges, dulling of the cutting edges and consequent degradation of cut quality or increased maintenance time for resharpening. As an alternative mechanical cutting method, high speed saw or abrasive cutting of fibrous pads can cause sufficient heat to be generated so that the dry reagents are affected and the adhesives used to bind the fibers are picked-up on the cutting tool, limiting its useful life. Since one would want to leave the space between pads as narrow as possible, while still preventing cross-over of liquids between adjacent pads, making sharp edges that define a gap between the pads is particularly important. The present invention addresses these problems by making accurate, straight cut edges at a controlled depth in a non-contact method at high speed.

### SUMMARY OF THE INVENTION

The present invention confines the spread of fluid which occurs upon application of sample to an indicating medium, such as a dry reagent on a paper carrier used for urinalysis. Specifically, lines are precisely scribed through a dry reagent and its carrier with a laser so that a narrow gap providing a physical barrier is created between reagent-containing pads, preventing fluid from spreading from one pad to another. The space between the reagent-containing pads is about 0.3 to 3 mm compared to the 12 to 14 mm spacing that has often been used.

### BRIEF DESCRIPTION OF THE DRAWINGS

FIG. 1 is an edge view of a test strip cut by a laser.

FIG. 2 illustrates an alternative embodiment of a reagent card of the invention.

### DESCRIPTION OF THE PREFERRED EMBODIMENTS

A current format for diagnostic dry reagents is a set of indicating ribbons adhered to a plastic card carrier. The ribbons are generally made of paper or other fibrous material mixed with dried reagent components. Each ribbon on the card constitutes a different reagent that is used to test for a specific analyte. Fluid sample is placed on a portion of each of the reagent ribbons in a column to test for the presence of multiple analytes in the sample, or for its physical properties. A subsequent fluid sample is placed on a neighboring portion of each of the ribbons in a separate column. Previously, the fluid samples placed on the ribbons had to be substantially separated (e.g. 12 to 14 mm) so that they did not bleed into the adjacent portion of the ribbon and mix with a different sample. By etching a narrow dividing space between adjacent portions of the ribbons, the fluid samples can be placed adjacent to one another without concern that the fluid will bleed into the adjacent portions of the ribbon.

Without having seen the present inventive method, one might conclude that using a laser to cut fibrous materials containing dried reagents might not be feasible. Not because of a laser's ability to cut the fibrous materials, but because of the heat generated by the cutting. The sensitive reagent potentially could be damaged by the laser's heat, as it separates the fibrous material. This could be expected to reduce the effective size of the pad as areas extending in from the edges became inactive or gave erroneous results. Also, if the residues of laser cutting remain on the cut edges the performance or at least the appearance could be degraded.

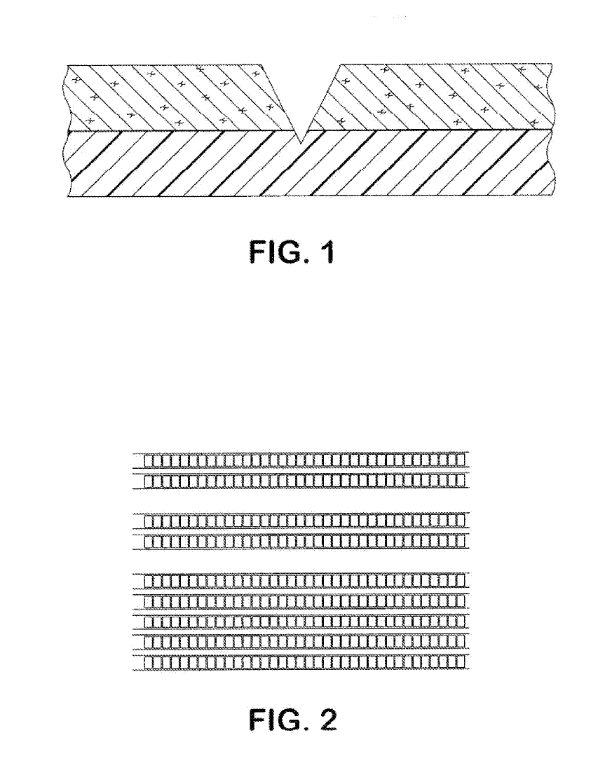

Despite the potential defects just discussed, a laser engraver was found to be an effective tool for scoring the ribbon by scribing lines in it only 0.1 mm wide. Specifically, a low power (20-120 watts) CO2 laser engraving system, such as those sold by Epilog Laser (Golden Colo.), was found suitable. Higher power laser systems would allow more rapid manufacture, but require more expensive laser systems. Ideally, all the fibrous material should be cut to form a gap sufficient to prevent the spread of fluid, while leaving the carrier intact. This prevents remaining strands of fibrous material from transferring fluid to an adjacent portion of the reagent ribbon. Laser etching or cutting was shown to produce such a gap while forming lines. FIG. 1 illustrates an edge view of a typical laser cut reagent-containing ribbon. The cut is very narrow as can be concluded from the ribbon and carrier thickness of only 0.69 mm. The laser cut narrows as the carrier is reached and it is estimated that the space separating the adjacent parts of the ribbon is only about 0.08 mm.

The lines could be scribed completely through the dry reagent ribbon medium and its carrier, but the laser technique has sufficient control to allow cutting the dry reagent layer without cutting through the carrier. Having the option to cut through the carrier or to cut all but the carrier layer allows flexibility of reagent format design and application. In FIG. 1, the laser has made a small cut in the carrier, which assures that no residual fibers remain in cut. The method of scoring reagent ribbon above the carrier achieves the fluid barrier, yet reduces the laser energy applied and so minimizes any damage to the reagent from the cutting process. Leaving the carrier intact or mostly intact can benefit in handling the card in automated instruments, for example in holding the card flat with a vacuum table.

Since the gap is narrow, it is preferred that the processed dry reagent-containing ribbon should not be supersaturated with sample. Adding too much sample to the border of the etched area of dry reagent can lead to leakage of sample across the etched border, and/or may interfere with the color reading.

The laser's ability to cut the ribbon medium is a proportional function of its power and speed. For a combination producing a particular cut depth, a lower power setting may be chosen, for example half the maximum available, but the time taken to make the cut will increase, to double in this example, so the speed of the cutting would also be halved. Generally, the thickness and material of the ribbon determines the ideal settings of power and speed that avoid injury to the reagents and reduction of the pad size. When the ribbon is cellulose paper an optimum combination of paper and speed would be determined, but glass fiber or other materials would require different settings for the same thickness. Other factors, such as composition and mass of dried reagent components within the ribbon also affect the ideal conditions, but the settings for manufacturing purposes remain constant for a reagent ribbon of controlled composition.

A typical laser etching system also operates at a predetermined and controlled frequency. A cut line is made by causing the ablation of tiny circular areas to overlap. If the frequency is set too low compared to the speed of the cut line, a series of ablated dots will form rather than a line of continuous scoring. Using a higher frequency than necessary to form a complete and continuous cut will form a line with very smooth edges, but will also apply more energy per unit distance of travel. An excess of energy applied could have undesirable effects, such as degradation of the appearance or performance of the reagent material close to the cut edge. In practice, the proper combination of percent power and speed for each reagent ribbon type would be determined first, then frequency would be adjusted so that microscope examination shows a continuous line of ablation. Then isolation of etched areas is confirmed by documenting wetting behavior when fluid is applied. The preferred combinations of power, speed and frequency which have been found for a group of reagent-containing ribbons appear in the table below:

| Reagent | Thickness | | | | 45 W MAX | |
|---|---|---|---|---|---|---|
| Ribbon | Material | Inches | mm | SPEED % | POWER % | Frequency |
| COL | Paper | 0.0280 | 0.711 | 80 | 20 | 400 |
| GLU | Paper | 0.0175 | 0.445 | 80 | 20 | 400 |
| BIL | Paper | 0.0330 | 0.838 | 100 | 20 | 500 |
| CRE | Paper | 0.0305 | 0.775 | 100 | 20 | 500 |
| PRO | Paper | 0.0200 | 0.508 | 100 | 20 | 500 |
| pH | Paper | 0.0170 | 0.432 | 100 | 20 | 500 |
| ALB | Glass Fiber | 0.0345 | 0.876 | 100 | 12 | 500 |
| OB | Paper | 0.0195 | 0.495 | 90 | 20 | 450 |
| KET | Paper | 0.0245 | 0.622 | 85 | 25 | 425 |
| URO | Paper | 0.0210 | 0.533 | 90 | 20 | 450 |
| NIT | Paper | 0.0380 | 0.965 | 80 | 25 | 400 |
| LEU | Paper | 0.0280 | 0.711 | 100 | 16 | 500 |

Where: COL=color; GLU=Glucose; BIL=Bilirubin; CRE=Creatinine; PRO=Protein; pH=pH; ALB=Albumin; OB=Occult Blood; KET=Ketone; URO=Urobilinogen; NIT—Nitrate; LEU=Leukocytes (white blood cells).

From these results, one can conclude that such a laser provides ample power for high speed etching of dry diagnostic materials.

A number of different etching (cutting) patterns may be used for isolating reaction regions. A top and bottom line may be scribed across the entire width of the ribbon so that a square is formed between the two-laser cut lines and the edge of the ribbon. Generally, a ribbon is 0.2 inches (5.1 mm) wide, so the laser-scribed lines would also be approximately 0.2 inches apart leaving reagent-containing pads with an area of 0.04 in² and a gap of about 0.1 to 0.3 mm. In a second embodiment, a rectangle or square area may be designated by scribing four lines (upper, lower, right and left) in the ribbon as shown in FIG. 2. This separated isolated reagent areas with a double line of etch. Although this embodiment was found to be effective, it left an undesirable amount of the ribbon unusable, in addition to requiring additional time and energy to scribe the additional lines. Further subdividing of the reagent pads is possible, that is, by cutting multiple concentric or parallel lines within the pads.

Advantages of the invention include reduced system size, increased throughput, and lower manufacturing costs. The concept allows a path toward smaller, faster, cheaper, better automated analysis. Current manufacturing methods can be used to prepare the cards of ribbons, a common intermediate for this concept and the current CLINITEK ATLAS rolls. From the card of ribbons, reaction areas can be defined by laser or mechanical cutting, which is much faster and less expensive than cutting the cards into strips and welding them to a roll of plastic carrier, the present CLINITEK ATLAS method. Use of laser etching in a manufacturing line provides other possible ancillary benefits to production efficiency. For example, the same laser system which scores the reagent ribbons could also be configured to cut the card sections from a roll of ribbons on a carrier or perhaps create the ribbons from wide reagent sheet stock.

Additionally, confining the spread of fluid allows a greater number of tests to be run per card, as less ribbon is contaminated by each application of fluid sample. In addition, the invention provides more uniform indication of the reagent response within the area of the diagnostic medium which is to be analyzed. It is surmised that reasons for this are that the surfaces of small areas are completely wet more rapidly than larger areas and that an area which is wet on the surface and soaks down through the reagent, treating each portion of the reagent area equally, while when fluid is applied to a large area it soaks downward and then migrates outward. If fluid is not confined, its spreading can be accompanied by undesirable effects, such as chromatography i.e. unevenness of color. In addition, the capability to place reaction areas closer together allows benefits, such as more compact assay format, less mass of used product biological waste, smaller instrument footprint and reduction of mechanical motions. Reducing motions required to perform assays can increase the throughput.

1. A diagnostic test strip comprising:
(a) a fluid impermeable substrate layer;
(b) a porous carrier adhered to said substrate;
(c) a liquid reagent solution impregnated and dried onto said porous carrier;
wherein said porous carrier with said impregnated and dried reagent was divided into reagent pads separated so as to prevent migration of a liquid sample between said reagent pads, said impregnated porous carrier being divided by removing portions of said test strip with a laser to form said reagent pads.

2. A diagnostic test strip of claim 1 wherein said reagent pads are separated by about 0.3 to 3 mm.

3. A diagnostic test strip of claim 1 wherein said reagent pads have an area of about 6 to 25 mm⁻².

4. A diagnostic test strip of claim 1 wherein the laser was operated at a combination of power, speed, and frequency to provide clean sharp edges of said carrier consistent with minimal heating of said reagent on said carrier.

5. A diagnostic test strip of claim 1 wherein said reagent pads are further divided by etching the edges of said pads between said portions removed with a laser.

6. A diagnostic test strip of claim 1 wherein said reagent pads are further divided by cutting through the substrate with a laser.

7. A diagnostic test strip of claim 1 wherein said reagent pads are further divided by multiple concentric or parallel etching with a laser.

8. A method of preventing migration of liquid samples on diagnostic test strips adhered to substrates comprising dividing diagnostic test strips into reagent pads by removing portions of said test strips with a laser.

9. A method of claim 8 wherein said laser is operated at a combination of power, speed, and frequency to provide clean sharp edges of said reagent pads consistent with minimal heating of said reagent pad.

10. A method of claim 8 wherein said test strips are divided into reagent pads spaced about 0.1 to 3 mm apart.

11. A method of claim 8 wherein said reagent pads have an area of about 6 to 25 mm⁻².

12. A method of claim 8 wherein said reagent pads are further divided by cutting through the substrate with a laser.

13. A method of claim 8 wherein said reagent pads are further divided by multiple or parallel etching with a laser.

14. A method of claim 8 wherein said reagent pads are divided by cutting through the substrate of said test strips with a laser.

15. A method of claim 8 wherein said reagent pads are further divided by multiple concentric or parallel etching with a laser to form the edges of said pads.

16. A diagnostic card having at least one ribbon adhered to a plastic card carrier, said diagnostic card comprising:
(a) a fluid impermeable substrate card;
(b) at least one porous carrier strip adhered to said substrate card;
(c) a liquid reagent solution impregnated and dried onto said porous carrier strip;
wherein said porous carrier with said impregnated and dried reagent was divided into reagent pads separated so as to prevent migration of a liquid sample between said reagent pads, said impregnated porous carrier being divided by removing portions of said test strip with a laser to form said reagent pads.

17. A diagnostic card of claim 16 wherein said reagent pads are separated by about 0.3 to 3 mm.

18. A diagnostic card of claim 16 wherein said reagent pads have an area of about 6 to 25 mm⁻².

19. A diagnostic card of claim 16 wherein the laser was operated at a combination of power, speed, and frequency to provide clean sharp edges of said carrier consistent with minimal heating of said reagent on said carrier.

20. A diagnostic card of claim 16 wherein said reagent pads are further divided by etching the edges of said pads between said portions removed with a laser.

21. A diagnostic card of claim 16 wherein said reagent pads are further divided by cutting through the substrate card with a laser.

22. A diagnostic card of claim 16 said reagent pads are further divided by multiple concentric or parallel etching with a laser.
